(19) United States
(12) Patent Application Publication
(10) Pub. No.: US 2018/0325605 A1
(43) Pub. Date: Nov. 15, 2018

(54) SYSTEM AND METHOD FOR PREVENTING WRONG SITE SURGERY

(71) Applicant: Safeside Surgical, LLC, Scarsdale, NY (US)

(72) Inventors: Douglas S. Scherr, Scarsdale, NY (US); Roger P. Goldberg, Evanston, IL (US); Michael Scherr, New York, NY (US); John V. Stone, Belmont, MA (US)

(21) Appl. No.: 15/975,565

(22) Filed: May 9, 2018

Related U.S. Application Data
(60) Provisional application No. 62/503,881, filed on May 9, 2017.

(51) Int. Cl. A61B 34/00; A61B 90/90; A61B 90/00; G16H 40/20

(52) U.S. Cl. CPC A61B 34/25; A61B 90/90; A61B 90/08; G16H 40/20; A61B 2034/254; A61B 2090/0807; A61B 2034/256; A61B 2090/0818; A61B 2034/258

(57) ABSTRACT

A method and system for confirming the siting of a surgical procedures prior to starting the procedure. The method is guided by a computer implemented surgery siting system operating in conjunction with at least one tangible component, such as a wearable device. The method and system are designed to confirm sidedness of a surgical procedure prior to initiating the surgical procedure through a series of checks and confirmations both by the computer implemented surgery siting system, and physical location of a wearable device containing machine readable data that correlates with the information accessible by the surgery siting system. The confirmation of the surgical siting is provided by following a series redundant and effective mechanisms that reduce the occurrence of wrong site surgeries.

### CROSS-REFERENCE TO RELATED APPLICATION(S)

This application claims priority to, and the benefit of, co-pending U.S. Provisional Application No. 62/503,881, filed May 9, 2017, for all subject matter common to both applications. The disclosure of said provisional application is hereby incorporated by reference in its entirety.

### FIELD OF THE INVENTION

The present invention relates to a system and method suitable for confirming surgical procedures prior to starting the procedure, confirming correct side of a procedure, and confirming patient identification. In particular, the present invention relates to a method guided by a software application working in conjunction with other tangible components designed to confirm sidedness and location of a planned surgical procedure as well as patient identification prior to initiating the surgical procedure.

### BACKGROUND

Generally, in the medical profession, wrong site surgeries are surgeries that should never occur but occur at an unacceptable rate. Wrong site surgeries include surgeries that are performed on the wrong part of a patient's body, the wrong surgery performed on the correct part of a patient's body, or the correct surgery performed on the wrong patient. Examples of an operation on the wrong side is the case of a patient who had the right kidney removed when there was a tumor on the left kidney. One example of surgery on the incorrect site is operating on the wrong level of the spine, a surprisingly common issue for neurosurgeons. A classic case of wrong-patient surgery involves a patient who undergoes a cardiac procedure intended for another patient with a similar last name. These types of wrong site surgeries are reported to occur 35,000 to 50,000 times a year.

There have been efforts in the medical community to prevent wrong site surgeries by developing mechanisms for identifying a correct site, procedure, and patient. For example, a common preventative mechanism of the “sign your site” methodology was implemented to mark the operative site on the patient in an unambiguous fashion (e.g., marking an “X” on the patient with a marker). The sign your site methodology was widely disseminated, however, implementation and adherence to the protocol differed significantly across medical facilities and specialties. Additionally, the sign your site often created confusion as to whether the marked site was intended to be avoided or indicated the site of the surgery. Other methods such as the universal protocol and pre-surgical checklists have also been implemented, however, wrong site surgeries still occur despite full adherence to these methodologies. Accordingly, an improved method and system is needed to reduce and/or eliminate the occurrence of wrong site surgeries.

### SUMMARY

There is a need for redundant and effective mechanisms that reduce wrong site surgeries. The present invention is directed toward further solutions to address this need, in addition to having other desirable characteristics. Specifically, the present invention is directed to a system that incorporates a specific combination of steps in combination with a specially designed software platform and tangible machine-recognizable code designed to reduce and/or eliminate the occurrence of wrong site surgeries. The specific combination of steps, provided by the present invention, amount to more than conventionally known techniques and steps merely implemented in a computer platform. Instead, the specific and unique combination of steps provided by the present invention were not previously implemented manually or implemented by a computer system. In particular, these steps provide a specific combination of steps using an unconventional combination of rules to enable an improved method for reducing wrong site surgery by medical practitioners.

In accordance with example embodiments of the present invention, a method for verifying a planned surgical procedure is provided. The method includes obtaining one or more medical scans or images of a patient, reviewing the one or more medical scans or images and selecting an apposite image to be uploaded for access by an application operating on a computing device. Patient information is supplied to the application in association with the apposite image, the patient information including an indication of sidedness of a procedure to be performed. The patient information is transformed into a scannable code, which is encoded with the patient information in a wearable device. The method further includes placing the wearable device on a limb of the patient corresponding to the sidedness of the procedure to be performed, performing a three step validation to confirm the procedure to be performed, and authorizing the procedure upon completion of the three step validation.

In accordance with aspects of the present invention, the patient information further comprises at least one of the procedure to be performed, a procedure code, a photo of the patient, a name of the patient, a medical record number of the patient.

In accordance with aspects of the present invention, the scannable code is a Quick Response Code (QR Code).

In accordance with aspects of the present invention, the three step validation includes a) instructing the patient to raise the limb with the wearable device and capturing a photographic image of the patient with the raised limb to verify placement of the wearable device on the limb of a same side of the sidedness of the procedure to be performed by checking whether a sidedness of the raised limb corresponds to the sidedness of the procedure to be performed, b) scanning the bar code on the wearable device and receiving back information comprising patient information and an indication of the sidedness of the procedure, and checking whether the indication of the sidedness of the procedure from the wearable device corresponds with the sidedness of the procedure to be performed to validate the sidedness of the procedure, and c) a user tapping the a touchscreen display of the apposite image presented on the application at a general location of the procedure as represented in the apposite image, and comparing the general location tapped with the sidedness of the procedure to be performed to validate the sidedness of the procedure to be performed. When the application confirms a correct validation for each step a) through c) of the three step validation, the application provides an authorization of the procedure to be performed. When the application confirms a correct validation for less than each step a) through c) of the three step validation, the application provides an indication of no authorization to proceed with the procedure to be performed.

In accordance with aspects of the present invention, the application is a cloud based application.

In accordance with aspects of the present invention, the confirming whether the sidedness of the raised limb corresponds to the sidedness of the procedure to be performed is performed through facial recognition software.

In accordance with an embodiment of the present invention, a method for verifying an intended or planned surgical procedure includes reviewing one or more obtained medical scans of a patient to select at least one apposite image. Patient information associated with the selected apposite image is received, the patient information including an indication of sidedness of a procedure to be performed. The patient information is transformed into a scannable code to be placed on a limb of the patient corresponding to the sidedness of the procedure to be performed. A validation is performed based on the scannable code placed on a limb of the patient corresponding to the sidedness of the procedure to be performed and the selected apposite image to confirm procedure to be performed. The procedure is authorized, or not, based on the performed validation.

In accordance with aspects of the present invention, a wearable device is created having the scannable code. The wearable device comprises a bracelet. The method can further include placing the wearable device on a limb of the patient corresponding to the sidedness of the procedure to be performed. The patient information can further include at least one of: the procedure to be performed, a procedure code, a photograph of the patient, a name of the patient, and a medical record number of the patient. The scannable code can be a Quick Response Code.

In accordance with aspects of the present invention, the validation can include performing a visual confirmation of patient, scanning the scannable code to verify patient information, and receiving, from a user, confirmation of sidedness of procedure to be performed.

In accordance with aspects of the present invention, performing visual confirmation can include taking a photograph of the patient holding up the limb with the scannable code indicating sidedness of the procedure to be performed, checking that the sidedness of the limb with the scannable code being held up in photo corresponds the sidedness of the procedure to be performed. Performing visual confirmation of patient can be performed using facial recognition software. The scanning of the scannable code to verify patient information can include identifying sidedness of procedure to be performed.

In accordance with aspects of the present invention, receiving, from a user, confirmation of sidedness for procedure to be performed can include displaying the selected apposite image can include receiving a selection of a location of the displayed selected apposite image from the user, and comparing the location of the selected apposite image selected by the user to sidedness of the procedure to be performed.

In accordance with embodiments of the present invention, a surgical siting system for verifying an intended or planed surgical procedure includes a memory for storing data and instructions for execution. A processor is provided, configured to review one or more obtained medical scans of a patient to select at least one apposite image. The processor is further configured to receive patient information associated with the selected apposite image, the patient information including an indication of sidedness of a procedure to be performed. The processor is configured to transform the patient information into a scannable code to be placed on a limb of the patient corresponding to the sidedness of the procedure to be performed. The processor is configured to perform a validation based on the scannable code placed on a limb of the patient corresponding to the sidedness of the procedure to be performed and the selected apposite image to confirm procedure to be performed. Further, the processor is configured to determine authorization of the procedure based on the performed validation.

In accordance with aspects of the present invention, the system can be further configured to create a wearable device having the scannable code. The patient information can further include at least one of: the procedure to be performed, a procedure code, a photograph of the patient, a name of the patient, and a medical record number of the patient.

The scannable code can be a Quick Response Code. The validation can include performing visual confirmation of patient, scanning the scannable code to verify patient information, and receiving, from a user, confirmation of sidedness of procedure to be performed.

In accordance with aspects of the present invention, the processor can be configured to perform visual confirmation to take a photograph of the patient holding up the limb with the scannable code indicating sidedness of the procedure to be performed, and check that the sidedness of the limb with the scannable code being held up in photo corresponds the sidedness of the procedure to be performed.

In accordance with aspects of the present invention, visual confirmation of patient is performed using facial recognition software. Configuring the processor to scan the scannable code to verify patient information can include configuring the processor to identify sidedness of procedure to be performed. Configuring the processor to receive, from a user, confirmation of sidedness for procedure to be performed can include configuring the processor to display the selected apposite image, receive a selection of a location of the displayed selected apposite image from the user, and compare the location of the selected apposite image selected by the user to sidedness of the procedure to be performed.

In accordance with embodiments of the present invention, a method for verifying a surgical procedure includes providing a surgical siting system comprising a processor and memory. A client device is provided having a processor and memory in communication with the surgical siting system. On the user device, one or more obtained medical scans of a patient are reviewed to select at least one apposite image. A selected apposite image is stored on the surgical siting system. On the user device, patient information associated with the selected apposite image is received, the patient information including an indication of sidedness of a surgical procedure to be performed. The patient information is stored on the surgical siting system. On the surgical siting system, the patient information is transformed into a scannable code to be placed on a limb of the patient corresponding to the sidedness of the procedure to be performed. Using the client device, a wearable device is generated having the scannable code provided by the surgical siting system. The wearable device is placed on a limb of the patient corresponding to the sidedness of the procedure to be performed. On the client device, a validation is performed based on the scannable code placed on a limb of the patient corresponding to the sidedness of the procedure to be performed and the selected apposite image provided by the surgical siting system to confirm procedure to be performed. On the client device, the procedure is authorized or not based on the performed validation.

In accordance with aspects of the present invention, the patient information further includes at least one of: the procedure to be performed, a procedure code, a photograph of the patient, a name of the patient, and a medical record number of the patient. The scannable code can be a Quick Response Code.

In accordance with aspects of the present invention, the validation can include performing, on the client device, visual confirmation of patient. On the client device, the scannable code can be scanned to verify patient information stored on surgical siting system. On the client device, confirmation of sidedness of procedure to be performed can be received from the user.

In accordance with aspects of the present invention, performing visual confirmation includes taking a photograph of the patient holding up the limb with the scannable code indicating sidedness of the procedure to be performed, and confirming that the sidedness of the limb with the scannable code being held up in photo corresponds the sidedness of the procedure to be performed. Performing visual confirmation of patient can be performed using facial recognition software. Scanning of the scannable code to verify patient information can include identifying sidedness of procedure to be performed stored on the surgical siting system. Receiving, from a user, confirmation of sidedness for procedure to be performed can include displaying, on the client device, the selected apposite image provided by the surgical siting system, receiving, on the client device, a selection of a location of the displayed selected apposite image from the user, and comparing the location of the selected apposite image selected by the user to sidedness of the procedure to be performed stored on the surgical siting system.

### BRIEF DESCRIPTION OF THE FIGURES

These and other characteristics of the present invention will be more fully understood by reference to the following detailed description in conjunction with the attached drawings, in which:

FIG. 1 is a diagrammatic illustration of a surgery siting system for implementation of the present invention;

FIG. 2 is an example login page for an application in accordance with aspects of the present invention;

FIG. 3 is an example wearable device in the form of a medical bracelet with a scannable code in accordance with aspects of the present invention;

FIG. 4 is an example patient profile page displayed by the application in accordance with the present invention;

FIG. 5 is an example photographic image display of a patient with the medical bracelet for use with the application in accordance with the present invention;

FIGS. 6A, 6B, and 6C are example displays of apposite images for use with the application in accordance with the present invention;

FIG. 7 is an example authorization page displayed by the application in accordance with the present invention;

FIGS. 8 and 9 are illustrative flowcharts depicting operation of a surgery siting device, in accordance with aspects of the invention; and

FIG. 10 is a diagrammatic illustration of a high level architecture of devices for implementing processes in accordance with aspects of the invention.

### DETAILED DESCRIPTION

An illustrative embodiment of the present invention relates to a system and method designed to prevent wrong site surgery. In particular, the present invention relates to a unique combination of steps implemented with a software application and additional tangible components designed to provide redundant confirmation mechanisms to prevent wrong site surgeries. The redundant mechanism involves following the specific combination of steps, as implemented through an application (e.g., a mobile application), to act as a check to approve a planned surgical procedure after confirming the procedure. Specifically, the medical professional is given authorization to proceed with a procedure upon confirmation of the patient, the site of the procedure, and the subject of the procedure, using the unique method and system of the present invention.

Initially, a patient meets with a medical professional (e.g., doctor, physician, surgeon, nurse, physician assistant, etc.) with a health issue resulting in the patient receiving image scans, blood work, and other tests required to form a diagnosis. When image scans (e.g., X-ray, computerized tomography (CT), magnetic resonance imaging (MRI), ultrasound, etc.) are obtained as part of a diagnosis, the medical professional reviews the image scans with the patient and identifies any areas of concern. When surgery or some other pre-defined invasive procedure involving “sidedness” is determined to be the proper course of treatment for the identified areas of concern, the medical professional begins utilizing the surgery siting system of the present invention. From all of the image scans, the medical professional will select at least one apposite image or key image to be uploaded for use by the surgery siting system. The at least one apposite image is determined to be a highly pertinent and appropriate image for depicting the area of concern and the intended target surgical site. With the apposite image uploaded, the medical professional enters patient information and procedure information, including a sidedness of the procedure (e.g., the side of the patient that the surgery will be performed), to be stored by the surgery siting system. It should be noted that medical professionals are generally accustomed to their own right side being their patient's left side when facing a patient. As such, the sidedness of a medical procedure is defined based on the patient's perspective or anatomical side of left or right side, not the medical professional's perspective.

The specific steps that make up the redundant confirmation mechanism of the present invention start on the day of the surgery or procedure during preparation of the surgical patient. Specifically, when the patient arrives at the medical facility (e.g., hospital, out-patient facility, etc.), the patient checks-in and receives a wearable device, such as a medical bracelet with a scannable code (e.g., bar code, quick response code (QR Code), etc.) printed thereon. The scannable code includes the patient information and procedure information (including the sidedness) previously recorded by the medical professional. The wearable device is placed on a limb of the patient (e.g., a wrist if it is a bracelet) corresponding with the side of the procedure being performed. For example, if the patient will be having the left kidney removed, the wearable device is placed on the left limb of the patient, e.g., on the left wrist, left forearm, or a finger of the left hand. During preoperative meeting with the patient, the medical professional performing the surgery must follow the redundant confirmation mechanism provided in the form of guidance by the surgery siting system.

Instructions are provided by the application requiring the medical professional to take a picture of the patient while the patient is holding the limb containing the wearable device up proximal to their face. The application confirms whether the limb being held help up corresponds to (e.g., matches) the correct sidedness of the intended or planned procedure. The medical professional is then instructed by the surgery siting system to scan the scannable code on the wearable device to reconfirm sidedness of the procedure. Lastly, the surgery siting system provides the medical professional with the previously uploaded apposite image scan and instructs the medical professional to select which side of the image that the procedure is to be performed. For example, a red line is superimposed over the apposite image down the center and when the medical professional touches the screen over the appropriate surgical site the system recognizes the selection as left or right of red line. The surgery siting system confirms whether that selection by the medical professional corresponds to the correct sidedness of the intended or planned procedure. Once all the confirmation mechanisms have been performed and verified to match the correct sidedness of the surgery, the surgery siting system indicates that the medical professional can proceed with the surgical procedure. If at any point along the steps of the method an incorrect match occurs, the surgery siting system indicates that the surgical procedure is not authorized.

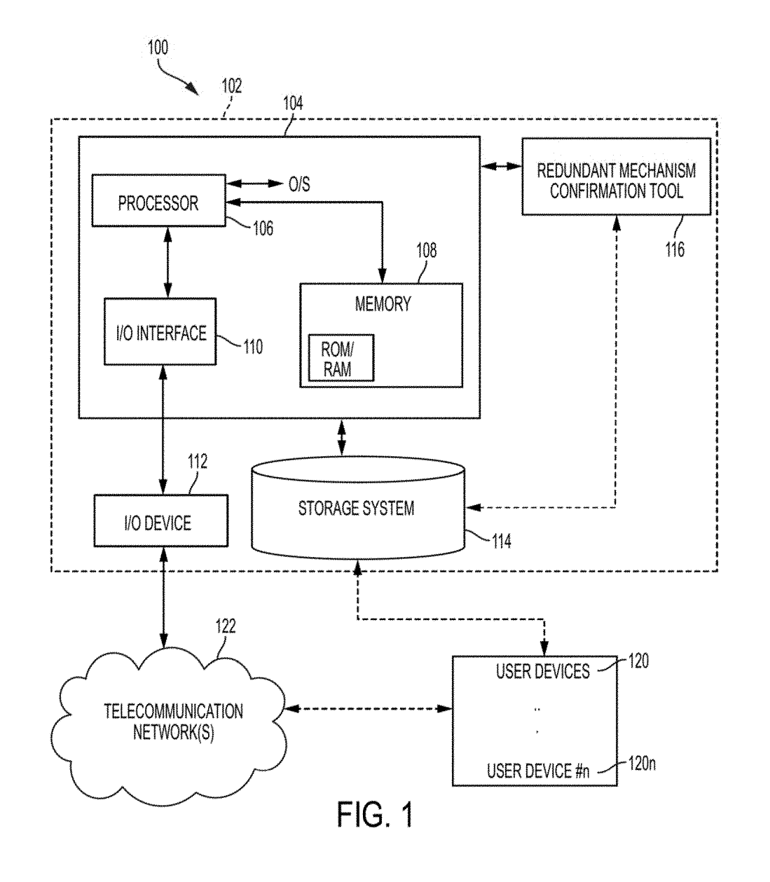

FIGS. 1 through 10, wherein like parts are designated by like reference numerals throughout, illustrate an example embodiment or embodiments of a unique method and system, with built-in redundancy, to prevent the occurrence of wrong site surgeries, according to the present invention. Although the present invention will be described with reference to the example embodiment or embodiments illustrated in the figures, it should be understood that many alternative forms can embody the present invention. One of skill in the art will additionally appreciate different ways to alter the parameters of the embodiment(s) disclosed in a manner still in keeping with the spirit and scope of the present invention.

FIG. 1 depicts an illustrative system 100 for implementing the steps in accordance with the aspects of the present invention. In particular, FIG. 1 depicts a system 100 including a surgery siting system 102. In accordance with an example embodiment, the surgery siting system 102 is a combination of hardware and software configured to carry out aspects of the present invention. In particular, the surgery siting system 102 can include a computing system with specialized software and databases designed for providing an application and method for preventing wrong site surgeries. For example, the surgery siting system 102 can be implemented in the form of software installed on a computing device 104, a web based application provided by a computing device 104 which is accessible by computing devices (e.g., the user devices 120), a cloud based application accessible by computing devices, or the like (e.g., via a mobile application).

In accordance with an example embodiment of the present invention, the surgery siting system 102 can include a computing device 104 having a processor 106, a memory 108, an input output interface 110, input and output devices 112 and a storage system 114. Additionally, the computing device 104 can include an operating system configured to carry out operations for the applications installed thereon. As would be appreciated by one skilled in the art, the computing device 104 can include a single computing device, a collection of computing devices in a network computing system, a cloud computing infrastructure, or a combination thereof. Similarly, as would be appreciated by one of skill in the art, the storage system 114 can include any combination of computing devices configured to store and organize a collection of data. For example, storage system 114 can be a local storage device on the computing device 104, a remote database facility, or a cloud computing storage environment. The storage system 114 can also include a database management system utilizing a given database model configured to interact with a user for analyzing the database data.

Continuing with FIG. 1, the surgery siting system 102 can include a combination of core components to carry out the various functions of the present invention. In accordance with an example embodiment of the present invention, the surgery siting system 102 can include a redundant mechanism confirmation tool 116. As would be appreciated by one skilled in the art, the redundant mechanism confirmation tool 116 can include any combination of hardware and software configured to carry out the various aspects of the present invention. In particular, each of the redundant mechanism confirmation tool 116 is configured to provide users with application protocol to implement the redundant confirmation mechanism to be carried out before authorization of a surgical procedure.

In accordance with an example embodiment of the present invention, the system 100 can include a plurality of user devices 120 configured to communicate with the surgery siting system 102 over a telecommunication network(s) 122. In particular, the system 100 includes a plurality of mobile user devices with a mobile application in communication with the surgery siting system 102 to carry out the specific aspects of the present invention. The surgery siting system 102 can act as a centralized host, for the user devices 120, providing the functionality of the redundant mechanism confirmation tool 116 sharing a secured network connection (e.g., meeting the health insurance portability and actability act (HIPAA) standards). As would be appreciated by one skilled in the art, the plurality of user devices 120 can include any combination of computing devices, as described with respect to the surgery siting system 102 computing device 104. For example, the computing device 104 and the plurality of user devices 120 can include any combination of servers, personal computers, laptops, tablets, smartphones, etc.

In accordance with an example embodiment of the present invention, the computing devices 104, 120 are configured to establish a connection and communicate over telecommunication network(s) 122 to carry out aspects of the present invention. As would be appreciated by one skilled in the art, the telecommunication network(s) 122 can include any combination of known networks. For example, the telecommunication network(s) 122 may be combination of a mobile network, Wi-Fi, WAN, LAN, or other type of network. The telecommunication network(s) 122 can be used to exchange data between the computing devices 104, 120, exchange data with the storage system 114, and/or to collect data from additional sources.

The redundant mechanism confirmation tool 116 is configured to provide a graphical user interface (GUI) to be displayed on the user devices 120. The GUI provides the functionality to the user device 120 to enable the required redundant confirmation mechanisms to be fulfilled by a medical professional to authorize proceeding with a surgical procedure. In particular, the GUI on the user device 120 is configured as the main interactive interface through which a medical professional interacts with the surgery siting system 102 (e.g., a cloud based system) to enable the redundant confirmation mechanism of the present invention. In accordance with an example embodiment of the present invention, the GUI is provided on a user device 120 through a user interface presented as part of a mobile application installed on the user device 120 or as part of a web interface presented on the user device 120 through another application (e.g., a web browser).

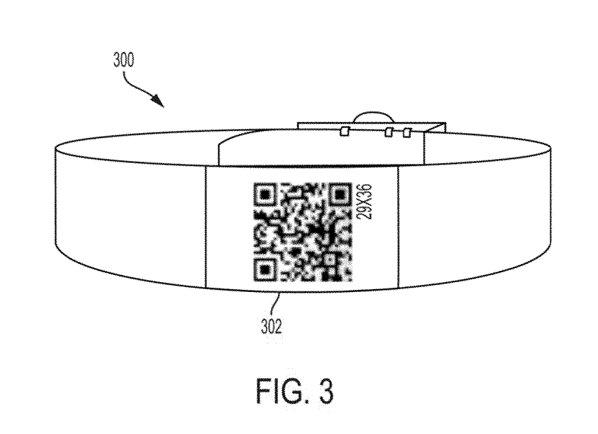

The system 100 further includes additional components including a tangible a wearable device 300 with a scannable code 302 (e.g., bar code, quick response code (QR Code), etc.) 302 displayed or provided thereon, as depicted in FIG. 3. The wearable device 300 in the illustrative example is depicted as a bracelet. However, one of skill in the art will appreciate the wearable device can take a number of different forms, with the ability to attach or couple to a limb of a patient, such as a wrap or cuff for a forearm, a bracelet for a wrist, or a ring for a finger, such that the present invention is not limited to the bracelet depicted in the figures.

The combination of hardware and software that make up the system 100 are specifically configured to provide a technical solution to a particular problem utilizing an unconventional combination of steps/operations to carry out aspects of the present invention. In particular, the system 100 leverages a software component in the form of the surgery siting system 102, which provides mechanisms and guidance resulting in redundancy checks to confirm the intended or planned surgical site and procedure, in combination with tangible components such as the wearable device that is physically worn by the patient and incorporates scannable data that interacts with the surgery siting system 102 software to provide additional redundancy and crosschecks to confirm the intended or planned surgical site and procedure. The overall system 100 is designed to execute a unique combination of steps to provide a novel approach to confirming a side of a surgical procedure, a site of a surgical procedure, a surgical procedure to be performed, and the patient in which the surgical procedure is to be performed upon.

In initial meetings, a patient with a health issue interacts with a medical professional (e.g., doctor, physician, surgeon, nurse, physician assistant, etc.). The patient may undergo various testing to determine the cause of the health issue. The various testing may include, but is not limited to, the medical professional performing imaging scans (e.g., X-ray, computerized tomography (CT), magnetic resonance imaging (MRI), ultrasound, etc.), blood work, and other tests required to form a diagnosis, all of which may produce one or more medical scans or images of the patient. The medical professional reviews the medical scans or images with the patient and identifies any areas of concern while determining possible courses of treatment. As would be appreciated by one skilled in the art, each of the testing and diagnostic testing is performed in the traditional manner as done in the medical profession. When a surgical procedure is determined to be the proper course of treatment for the identified areas of concern, the medical professional can begin utilizing the system 100 of the present invention by loading data, scans, images, etc. into the surgery siting system 102.

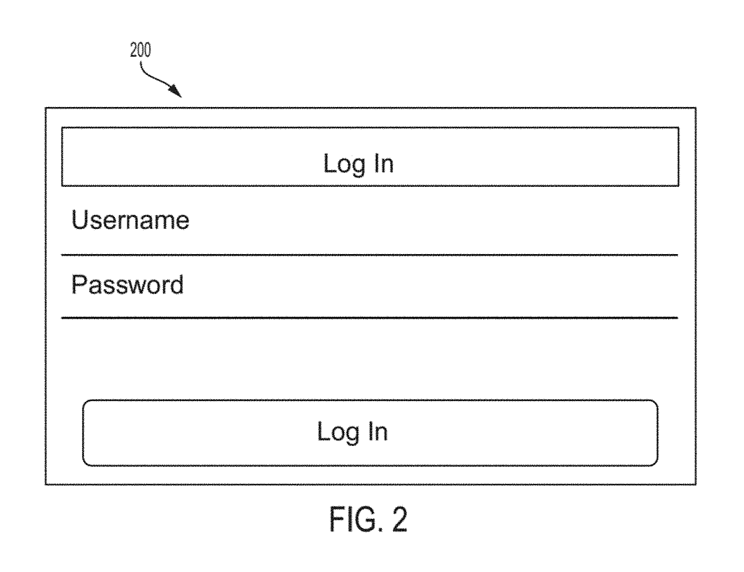

In accordance with an example embodiment of the present invention, storage of patient information, access to patient information, and implementation of the redundant confirmation mechanisms are provided by the surgery siting system 102 through a GUI, which can be provided on the user device 120 or can be through other peripheral computing devices accessing the surgery siting system 102 via cloud computing or other computer network structures. Initially, the surgery siting system 102 requires a medical professional to securely login to the surgery siting system 102. This login can be done, for example, using a GUI provided by a mobile application operating on the user device 120, which includes a login screen 200 for the medical professional. As would be appreciated by one skilled in the art, the login screen 200 can include a combination of a Username and Password for the medical professional to securely login and access the features of the surgery siting system 102, as depicted in FIG. 2. Additionally, the login screen 200 and medical professional login can be configured to be in compliance with HIPPA standards. Once logged into the surgery siting system 102 through the user device 120, the medical professional has access to the storage system 114 of the surgery siting system 102 and can add and/or modify data for the patient (e.g., within patient profiles). Again, the medical professional can use a mobile application operating on a user device 120, or can use, e.g., an office desktop computer in a patient examination room or doctor's office, to access the surgery siting system 102 and provide the data and information.

To initialize setup of redundant confirmation mechanisms, the surgery siting system 102 requires that the medical professional add and confirm the patient information to a patient profile and upload at least one apposite image 206 from any available image scans to the storage system 114. As would be appreciated by one skilled in the art, the patient information can include any necessary information required to provide medical services to the patient. For example, the patient information can include name, date of birth, social security number, blood type, known allergies, health insurance, medical record number, etc. Additionally, the patient information can include taking a picture of the patient with the user device 120, or other digital camera, and saving the picture to the patient profile within the surgery siting system 102 for identification purposes.

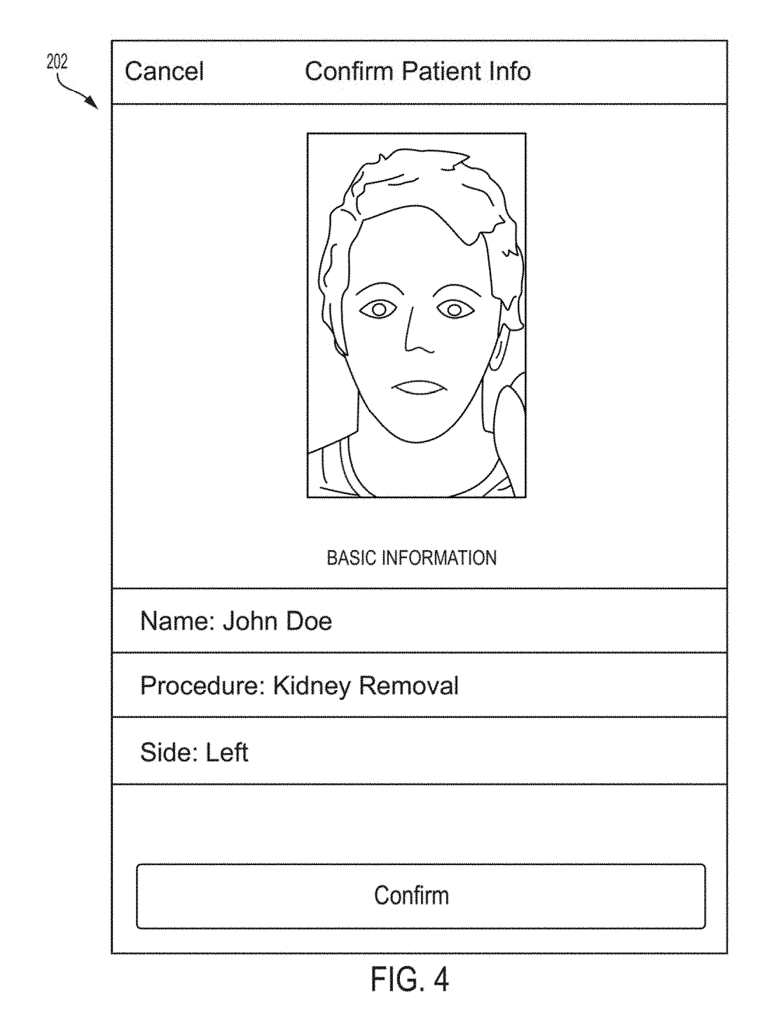

In accordance with an example embodiment of the present invention, the patient information includes a procedure or surgery to be performed and the sidedness of the procedure to be performed, which in combination define the intended or planned surgical procedure). In particular, the medical professional must indicate which side of the patient or part of the patient that the medical procedure is to be performed. For example, if a patient is having a left kidney removed, then the medical professional will indicate “left side” to the surgery siting system 102, via the user device 120 or other computer access point. The patient information is uploaded to the surgery siting system 102, and stored remotely within the storage system 114 of the surgery siting system 102. The information stored within the patient profile on the surgery siting system 102 can be provided back to the medical professional at any point in time by accessing the patient profile on the GUI of the user device 120, or on another computing access point such as a doctor's office computer, as depicted in FIG. 4.

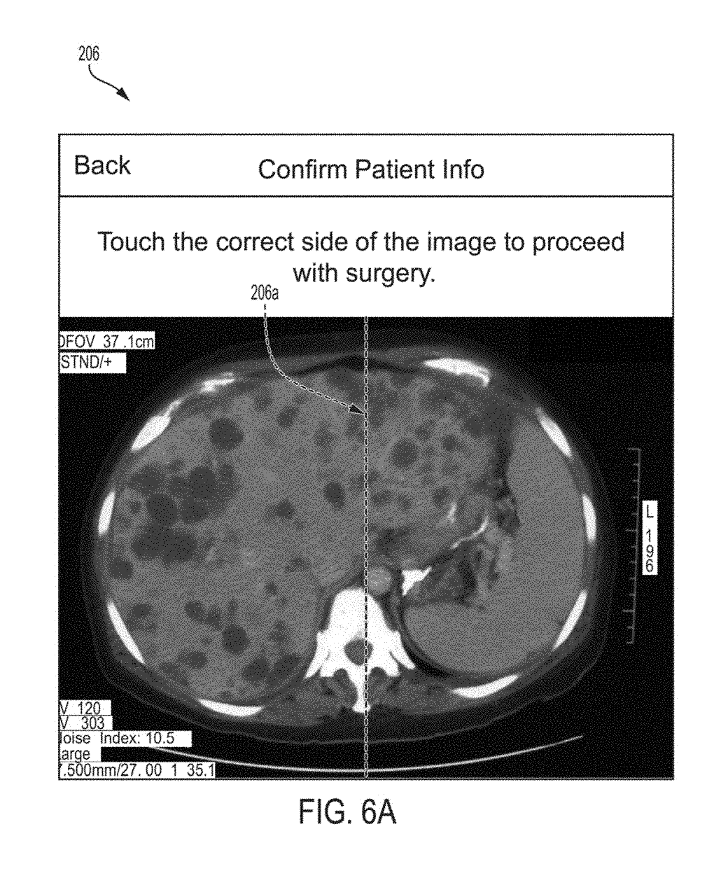

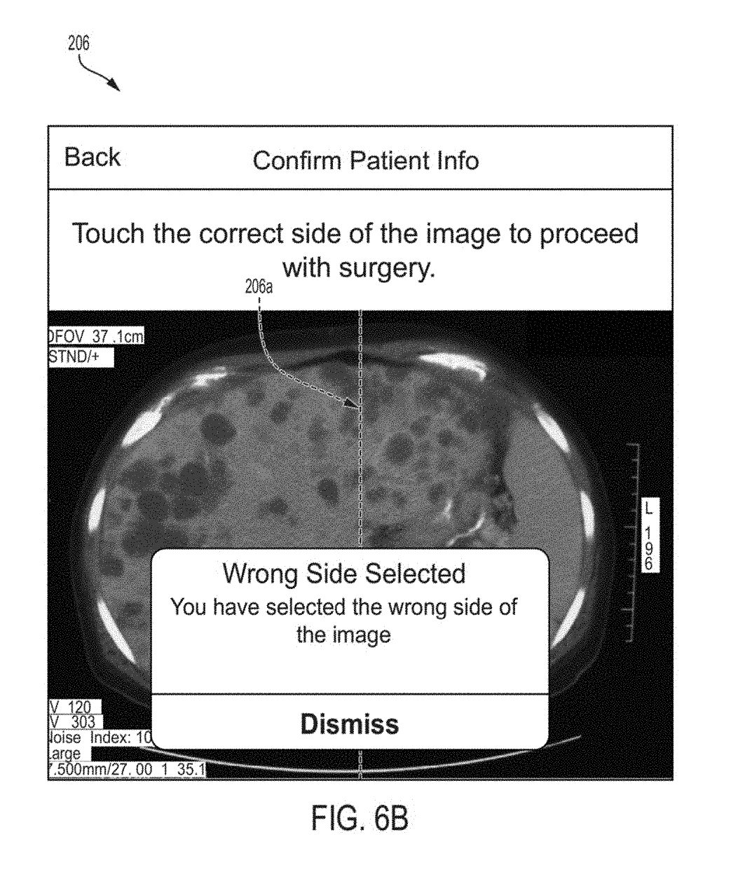

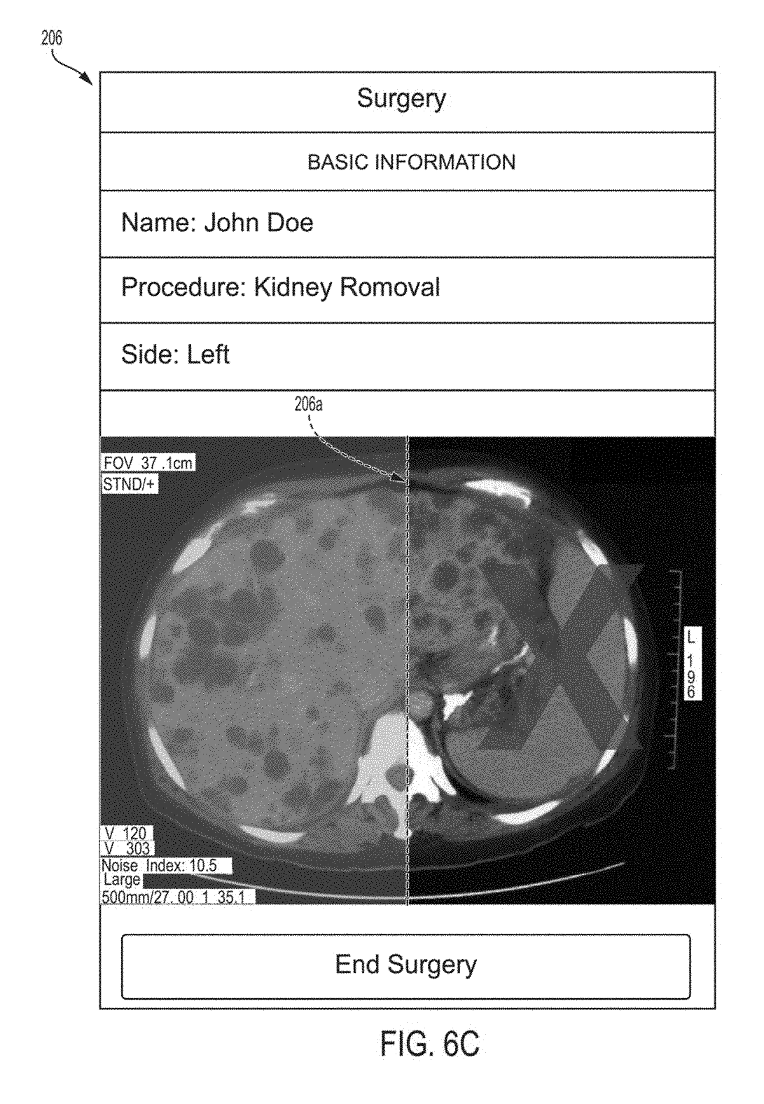

In accordance with an example embodiment of the present invention, the apposite image 206 to be uploaded must meet predetermined criteria, which can be outlined by the surgery siting system 102 for the medical professional to review. For example, the apposite image 206 to be uploaded to the surgery siting system 102 must include at least two identifiable hemispheres or sides of the object(s) and/or patient. The at least two identifiable hemispheres or sides of the object(s) and/or patient can further be differentiated by some combination of the application and/or the medical professional. For example, the surgery siting system 102 can display the apposite image 206 to the medical professional with an overlay or filter to be applied thereto to differentiate the two sides of the depicted image, scan, and/or patient. This overlay or filter may be as simple as a dotted line 206a separating two sides of the image, as depicted in FIGS. 6A-6C. Thereafter, the apposite image 206, including a selection of the sidedness of the procedure (e.g., the side of the patient or part of the patient on which the surgery will be performed), is uploaded and saved to the surgery siting system 102 (e.g., within the patient profile stored within the storage system 114)Upon completion of this step, the patient information and the apposite image 206 are both stored at the storage system 114 of the surgery siting system 102 for retrieval and utilization at a later point in time (e.g., the day of the surgery or medical procedure) with the redundant confirmation mechanism.

The specific steps that make up the redundant confirmation mechanisms of the present invention are carried out by the medical professional through the GUI operating on a user device 120 at a predetermined point in time. In accordance with an example embodiment of the present invention, the predetermined point in time is on the date of the surgery or medical procedure. When the patient arrives at the medical facility (e.g., hospital, out-patient facility, etc.), on the day of the surgery or medical procedure, the patient will check-in and receive a wearable device 300 with a scannable code 302 (e.g., bar code, quick response code (QR Code), etc.) 302 displayed or provided thereon, as depicted in FIG. 3. The wearable device 300 in the illustrative example is depicted as a bracelet. However, one of skill in the art will appreciate the wearable device can take a number of different forms, with the ability to attach or couple to a limb of a patient, such as a wrap or cuff for a forearm, a bracelet for a wrist, or a ring for a finger, such that the present invention is not limited to the bracelet depicted in the figures.

Continuing with the method, when patient arrives, a medical professional (e.g., nurse, receptionist, etc.) confirms the identity of the patient (e.g., via a license, passport, etc.) and will utilize the GUI on the user device 120 to accesses the patient profile (e.g., including the patient information and images associated therewith) that is stored in the surgery siting system 102. In particular, the medical professional will utilize the user device 120 to login to the surgery siting system 102 (e.g., via login screen 200) and select a patient profile stored within the storage system 114 of the surgery siting system 102. In accordance with an example embodiment of the present invention, the surgery siting system 102and/or medical professional can utilize the picture of the patient stored in the patient profile, as shown in FIG. 4, as confirmation of the identity of the patient to undergo the procedure.

Upon confirmation of the patient identity, the medical professional can select an option within the GUI on the user device 120 to create the wearable device 300 with the scannable code 302 for the patient to wear. The scannable code 302 includes the patient information and procedure information (including the sidedness) previously stored within the storage system 114 by the medical professional embedded therein. As would be appreciated by one skilled in the art, the wearable device 300 can include any combination of information utilized by the medical facility.

In accordance with an example embodiment of the present invention, the GUI on the user device 120 presents the medical professional with a confirmation patient information screen 202. The patient information screen 202, as depicted in FIG. 4, includes the basic information of the patient (e.g., John Doe), the procedure being performed on that day (e.g., kidney remove), and the sidedness of the procedure (e.g., left side). Once the medical professional confirms the identity of the patient (e.g., checking identification of the patient, comparing the saved photo of the patient to the patient, etc.), the wearable device 300, as depicted in FIG. 3, is created and placed on the patient (e.g., as prompted by the application). In accordance with an example embodiment of the present invention, the wearable device 300 in the form of a bracelet or other device is placed on the limb of the patient corresponding to the side of the procedure being performed. For example, if the patient will be having their left kidney removed, the wearable device 300 is placed on the left limb of the patient. The application indicates to the medical professional which limb to place the wearable device 300 on of the patient based on the sidedness information previously received by the application, as depicted in FIG. 4.

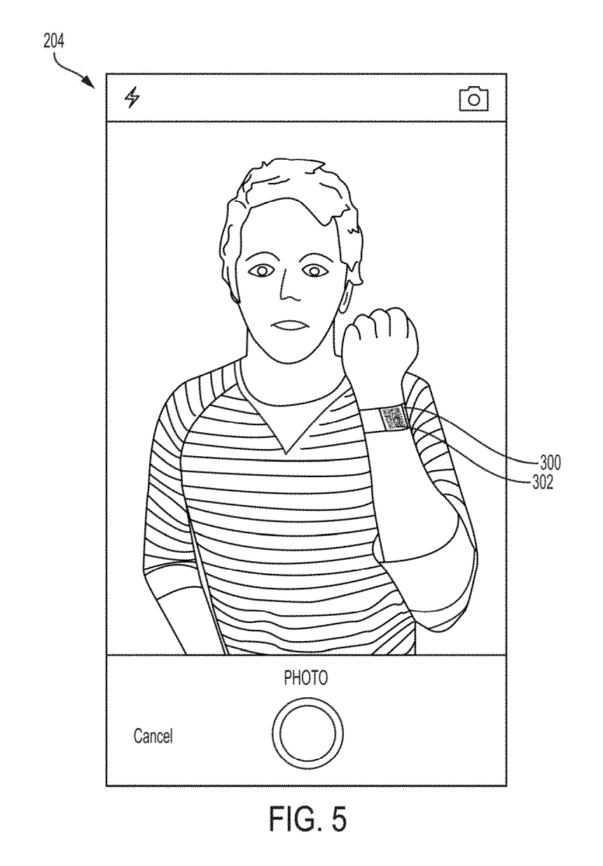

Once the wearable device 300 has been placed on the patient and placement of the wearable device 300 is confirmed by the medical professional on the user device 120, the surgery siting system 102 receives the confirmation and initiates the redundant confirmation mechanism for preventing wrong site surgeries. The first mechanism includes the surgery siting system 102instructing the medical professional, through the GUI on the user device 120, to take a photographic image 204 of the patient while the patient is holding the limb containing the wearable device 300 (e.g., their arm) proximal to their face, such as is depicted in FIG. 5. For example, the user device 120 can be utilized to capture the photographic image 204 (e.g., via smartphone camera) and upload the photographic image 204 to the surgery siting system 102for additional processing. As would be appreciated by one skilled in the art, the medical professional can utilize any image capturing device capable of sharing (e.g., via wire, Bluetooth, Wi-Fi, etc.) the image with the surgery siting system 102of the present invention.

Once the photographic image 204 is uploaded from the user device 120, the surgery siting system 102 confirms whether the limb being held up by the patient, as reflected in the photographic image 204, corresponds to the correct side of the intended or planned procedure (e.g., the sidedness indicated in the stored patient information). In accordance with an example embodiment of the present invention, the surgery siting system 102 utilizes facial recognition technology to identify the face of the patient in the photographic image 204 and identify the location of the limb of the patient in relation to the face. Once the location of the limb is identified, the surgery siting system 102 can determine whether the limb being held proximal to the patient's face corresponds to the side of the patient in which the intended or planned procedure is to be performed (e.g., per the sidedness stored in the surgery siting system 102). For example, continuing the above example, the surgery siting system 102 determines whether the patient is holding up their left arm in the photographic image 204. As would be appreciated by one skilled in the art, the surgery siting system 102 will recognize that the picture is an inverted sidedness of the actual sidedness of the patient in reality and make the determination accordingly. For example, using the photographic image 204 in FIG. 5 surgery siting system 102 will recognize that the patient is holding up their left wrist and respond accordingly. Continuing the example, since the patient is holding up the left wrist with the medical bracelet wearable device 300 thereon, then the surgery siting system 102 (e.g., the redundant mechanism confirmation tool 116), will indicate a pass (i.e., an approval) of this stage of the redundant confirmation mechanisms. The surgery siting system 102 will indicate confirmation of the correct limb to the user device 120 to be displayed to the medical professional.

Continuing to the next mechanism, the surgery siting system 102 transmits instructions to the medical professional, through the GUI on the user device 120, to scan the scannable code 302 on the wearable device 300, as depicted in FIG. 3. The medical professional utilizes the user device 120 to scan the scannable code 302 and upload the data from the scan to the surgery siting system 102 to confirm sidedness of the procedure. For example, the user device 120 can be utilized to scan the scannable code 302 (e.g., via smartphone camera) and provide the scanned information to the surgery siting system 102 for additional processing. As would be appreciated by one skilled in the art, the medical professional can utilize any barcode scanning device capable of scanning the scannable code 302 and sharing (e.g., via wire, Bluetooth, Wi-Fi, etc.) the information from the scannable code 302 with the surgery siting system 102.

In accordance with an example embodiment of the present invention, the surgery siting system 102can automatically derive the information from the scannable code 302 from the photographic image 204 of the patient with the wearable device 300 held proximal to the patient's face. This step replaces the need to manually scan the scannable code 302 on the medical bracelet wearable device 300 and upload the scanned data to the surgery siting system 102. Additionally, the surgery siting system 102 can utilize facial recognition technology can compare the face of the patient in the photographic image 204 with the picture of the patient stored in the patient profile to confirm the identity of the patient.

Lastly, the final mechanism includes instructing the user device 120 to display the previously uploaded apposite image 206 to the medical professional with instructions to select the side of the apposite image 206 that the procedure is to be performed upon (as understood by the medical professional). The medical professional uses the user device 120 to select one of the sides of the apposite image 206 or portion of the apposite image 206 indicating the side that they understand is the side to be operated upon. For example, the medical professional can select one side of the apposite image 206 on a touch screen of the user device 120. Once the medical professional makes a selection on the user device 120, the user device 120 uploads the selection to the surgery siting system 102 for analysis.

In accordance with an example embodiment of the present invention, the medical professional performing the selection is the medical professional performing the procedure. Based on the uploaded selection, the surgery siting system 102 determines whether the selection of the medical professional corresponds to the correct side of the procedure, as previously indicated by the medical professional (e.g., the stored sidedness value). If the surgery siting system 102 determines that the medical professional has selected the wrong side (e.g., continuing the example, the right side), the surgery siting system 102 provides instructions to the user device 120 to indicate that the wrong side was selected and deny approval for the procedure (e.g., via the GUI on the user device 120), as shown in FIG. 6B. If the surgery siting system 102 determines that the medical professional has selected the correct side (e.g., continuing the example, the left side) the surgery siting system 102 provides instructions to the user device 120 to indicate that the correct side was selected and approve for the procedure (assuming that each of the previous mechanisms also were positive), as shown in the GUI depicted in FIG. 6C.

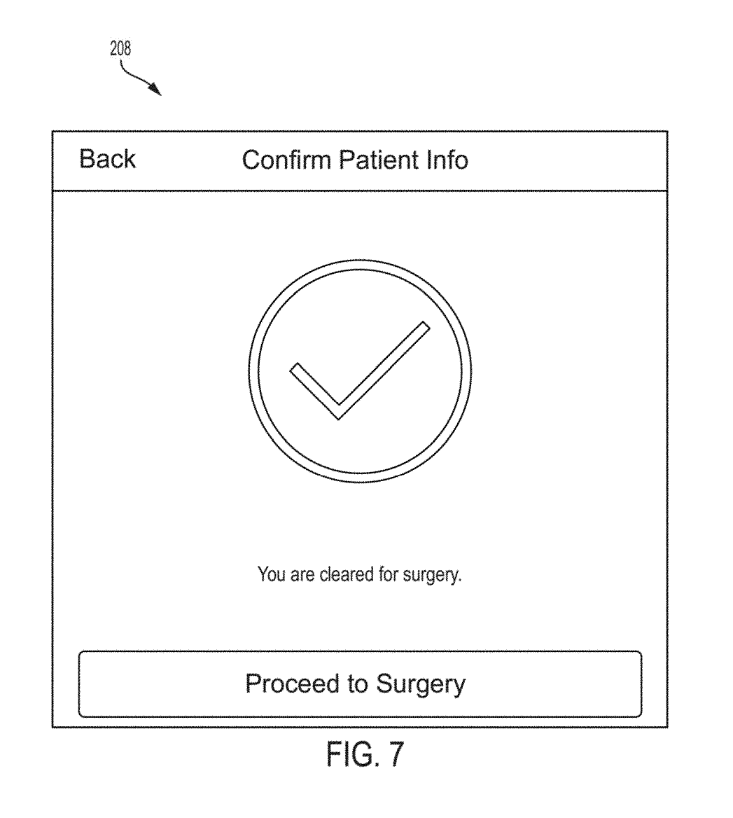

Additionally, the GUI provided on the user device 120 represented in FIG. 6C, will provide a final confirmation of the patient, the procedure, and what portion of the apposite image 206 that the procedure is to be performed upon. Once all of the confirmation mechanisms have been performed and verified, by the surgery siting system 102, to match the sidedness of the procedure, the surgery siting system 102 instructs the user device 120 to display an approval screen 208 to indicate that the medical professional can proceed with the surgical procedure, as shown in FIG. 7.

In accordance with an example embodiment of the present invention, each of the redundant confirmation mechanisms and the medical professional(s) response thereto is recorded and tracked by the surgery siting system 102. For example, each time that a medical professional incorrectly identifies a side of an apposite image 206, the surgery siting system 102 stores and tracks the results. The tracked information can include the redundant confirmation mechanism, the response to the redundant confirmation mechanism, and the time at which the selection was made. As would be appreciated by one skilled in the art, although the present invention is discussed with respect to the surgery siting system 102 performing the primary analyses, determinations, and computations throughout the redundant confirmation process while exchanging data with the user device 120, the user device 120 can be configured to perform all the analyses, determinations, and computations discussed with respect to the surgery siting system 102 without communicating to a remote system. For example, the user device 120 can include a standalone program and storage for carrying out the various aspects of the present invention.

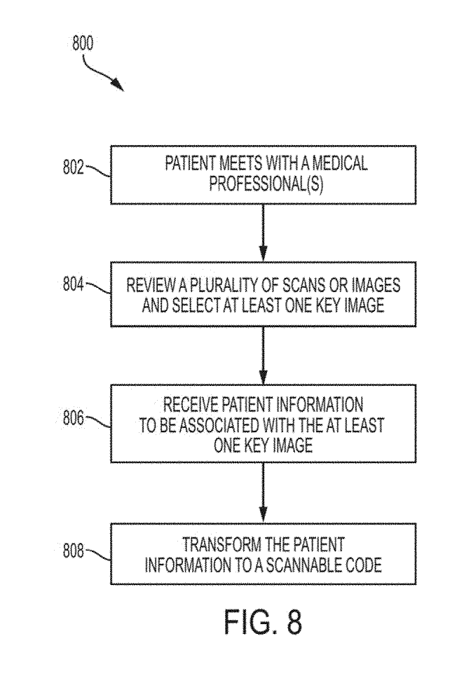

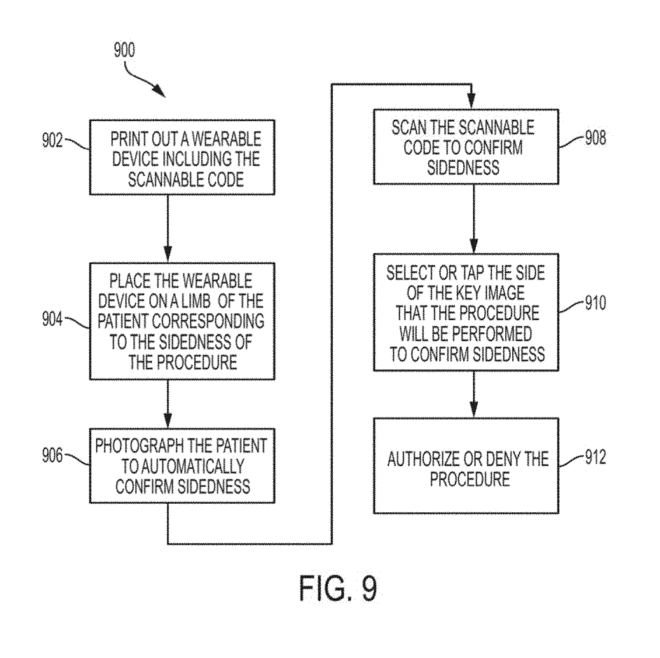

FIGS. 8 and 9 show exemplary flow charts depicting implementation of the present invention. Specifically, FIGS. 8 and 9 depict exemplary flow charts showing the computer implemented method for verifying a surgical procedure, as discussed with respect to FIGS. 1-7. In particular, FIG. 8 depicts the consultation process 800 performed prior to the day of surgery and FIG. 9 depicts the preoperative process 900 performed on the day of surgery.

The consultation process 800 starts at step 802 when the patient meets with a medical professional prior to the date of the surgery performed at a later date. In accordance with an example embodiment, prior to initiation of the consultation process 800, the medical professional performs testing, diagnostic procedures, and captures or otherwise obtains medical scans or images of the patient. For example, the medical professional can order X-ray images, CT scan, ultrasound scans, etc. of the patient in an effort to identity a medical issue and the corresponding course of action (e.g., surgery). At step 804 the medical professional will access and review the medical scans or images and select at least one apposite image 206 to be uploaded to the surgery siting system 102. For example, the medical professional can locally review a plurality of medical scans or images of the patient on an application installed as a mobile application on a user device 120 (e.g., smartphone, tablet, office computer, etc.) and select at least one preferred or apposite image 206 to be uploaded to a storage system 114 associated with the surgery siting system 102 of the present invention (e.g., via a web interface, mobile application, etc.). As would be appreciated by one skilled in the art, the plurality of images can be uploaded to the user device 120 or otherwise displayed on the user device 120 through remote access to a medical scan/image database of utilizing any other method or system known in the art. The one preferred or apposite image 206 will be saved by the surgery siting system 102 in a patient profile for utilization during the consultation process 800 and the preoperative process 900.

At step 806 the medical professional enters patient information into the surgery siting system 102. As would be appreciated by one skilled in the art, the medical professional can indicate the sidedness of the procedure through any combination of inputs. For example, the medical professional can select the side from a drop down, type in the side, and/or touch the appropriate side of the least one apposite image 206 itself on a touch screen of the user device 120. The patient information can also include the procedure to be performed, a procedure code, a photo of the patient, a name of the patient, or a medical record number of the patient. Once the patient information has been entered in the surgery siting system 102, the patient information is associated with the patient profile and the at least one apposite image 206 and stored within the storage system 114 of the surgery siting system 102 for later retrieval.

At step 808 the surgery siting system 102 transforms the patient information to a scannable code 302 to be associated with the patient profile and stored in the storage system 114. For example, the surgery siting system 102 encodes the patient information into a scannable QR Code and stores the QR Code with the patient profile. Once the at least one apposite image 206 has been uploaded, the patient information (including the sidedness of the surgery) has been uploaded, and the scannable code 302 has been created, the patient profile is configured for utilizing during the preoperative process 900.

FIG. 9 depicts steps included within the preoperative process 900 to be carried out by the medical professional(s) on the day of surgery. In particular, FIG. 9 depicts the preoperative process 900 for implementing the redundant confirmation mechanisms of the present invention for authorization to proceed with a surgical procedure. The preoperative process 900 takes place after the patient has already checked-in with the medical facility on the day of surgery. As would be appreciated by one skilled in the art, checking-in includes properly identifying the patient and locating a patient profile (from the surgery siting system 102) associated with the identified patient on a user device 120. Once the patient has been identified, a medical professional can login to the surgery siting system 102 using a user device 120 (e.g., via login screen 200) to start the preoperative process 900 and execute the redundant confirmation mechanisms in preparation for the surgical procedure.

At step 902 the medical professional uses the user device 120 to request a wearable device 300 including the scannable code 302 encoded with the patient information. For example, the medical professional selects a print option on the GUI of the user device 120 to print out a medical bracelet including a scannable code 302. In particular, the patient profile in the surgery siting system 102 will provide the medical provider with an option to print the medical bracelet including the scannable code 302 encoded with the information stored within the patient profile of the storage system 114. The scannable code 302 being the scannable code 302 created in step 808 of consultation process 800.

At step 904 the medical professional is instructed by the surgery siting system 102, via the user device 120, to place the wearable device 300 on a limb of the patient corresponding to the sidedness of the procedure to be performed. In particular, the GUI on the user device 120 instructs the medical professional to place the wearable device 300 on the limb of the patient corresponding to the sidedness indicated in the patient profile (e.g., as indicated at step 806). The surgery siting system 102 can also seek confirmation from the medical professional that the wearable device 300 has been placed. For example, the surgery siting system 102 can request that the medical professional indicate on the GUI of the user device 120 when the wearable device has been placed on the patient.

Steps 906-910 include the redundant confirmation mechanisms (or three step validation process) required to confirm the sidedness of the procedure to be performed. In particular, steps 906-910 include the steps to ensure that the surgical procedure is performed on the right part at the right location. At step 906 the surgery siting system 102 provides instructions to the user device 120 to instruct the medical profession have the patient raise the limb with the wearable device 300 attached thereto. Thereafter, the surgery siting system 102 instructs the medical professional, via the user device 120, to capture and upload a photographic image 204 of the patient with the raised limb to verify placement of the wearable device 300 on the limb of the correct sidedness of the intended or planned procedure to be performed. After receiving the upload of the photographic image 204 from the user device 120, the verification is performed by the surgery siting system 102 by comparing a sidedness of the raised limb with the previously stored sidedness of the procedure to be performed (e.g., stored in the patient profile). Optionally, the confirmation of the patient identity and auto scanning of the scannable code 302 are also performed as part of this step instead of at step 908.

Continuing with step 906, the surgery siting system 102 performs an automatic confirmation as to whether the wearable device 300 is in fact placed on the correct limb of the patient. In particular, the surgery siting system 102 detects the raised wrist of the patient and the face of the patient (e.g., through facial recognition technology) in the photographic image 204. Based on the identification of the raised limb and face of the patient, the surgery siting system 102 checks whether the appropriate limb is raised corresponding to the sidedness of the operation to be performed. For example, continuing the example, the surgery siting system 102 analyzes the photographic image 204 to determine whether the patient is holding up the left wrist (e.g., the raised wrist is located to the right of the face of the patient). If the surgery siting system 102 determines that the appropriate limb is raised, then the first of the redundant confirmation mechanisms is passed (i.e., approved). Otherwise the surgery siting system 102 indicates to the medical professional, via the user device 120, that the wearable device 300 is placed on the wrong limb and repeats steps 904-906 until the wearable device 300 is located on the correct limb before proceeding to step 908.

At step 908 the surgery siting system 102 instructs the medical professional, via the user device 120, to scan and upload the scannable code 302 embedded on the wearable device 300. Based on the uploaded data from the scannable code 302, the surgery siting system 102 derives an indication of the sidedness of the procedure from the scannable code. Thereafter, the surgery siting system 102 checks whether the indication of the sidedness of the procedure from the wearable device 300 corresponds with the sidedness of the procedure to be performed according to the patient profile. Additionally, the application compares the sidedness of, as indicated by the scannable code 302, against the sidedness confirmed in step 906. If the application determines that the appropriate sidedness is indicated on scannable code 302 of the wearable device 300, then the second of the redundant confirmation mechanisms is passed. Otherwise the surgery siting system 102 indicates to the medical professional, via the user device 120, that the wearable device 300 indicates a different sidedness from the sidedness stored in the patient profile and that proper correction is required before proceeding to step 910. Additionally, if the wearable device 300 indicates a different sidedness from the sidedness stored in the patient profile, then the surgery siting system 102 issues a warning with instructions of “DO NOT PROCEED”. At this point a check list of potential problems would be conveyed to the medical professional to examine and correct the problem.

At step 910 the surgery siting system 102 retrieves the previously stored apposite image 206 (e.g., step 804) and transmits the apposite image 206 to the medical professional on the user device 120 for display, as depicted in FIG. 6A. Thereafter, the surgery siting system 102 instructs the medical professional, via the user device 120, to select or tap the side of the apposite image 206 that the procedure will be performed. For example, the medical profession can tap a touchscreen display of the user device 120 displaying the apposite image 206. As part of the redundant confirmation mechanisms, the surgery siting system 102 compares the location selected by the medical profession on the user device 120 with the sidedness of the procedure to be performed according to the patient profile and/or the scannable code 302. If the application determines that the appropriate side of the apposite image 206 (e.g., the left side) was selected, then the last of the redundant confirmation mechanisms is passed. Otherwise the surgery siting system 102 indicates to the medical professional, via the user device 120, that the wrong side of the apposite image 206 was selected and repeats step 910 until the correct side of the apposite image 206 is selected before proceeding to step 912.

At step 912, if all of the redundant confirmation mechanisms in steps 906-910 are passed, then the surgery siting system 102 instructs the user device 120 to display authorization of the procedure, as shown in FIG. 7. Upon confirmation the medical professional can optionally mark the patient (e.g., using a marker) and transport the patient to the operating room. As would be appreciated by one skilled in the art, each of the steps in processes 800 and 900 do not have to be performed by a single medical professional. For example, a surgeon can perform steps 802-808 and steps 908-910 while a nurse or medical assistant can perform steps 902-904.

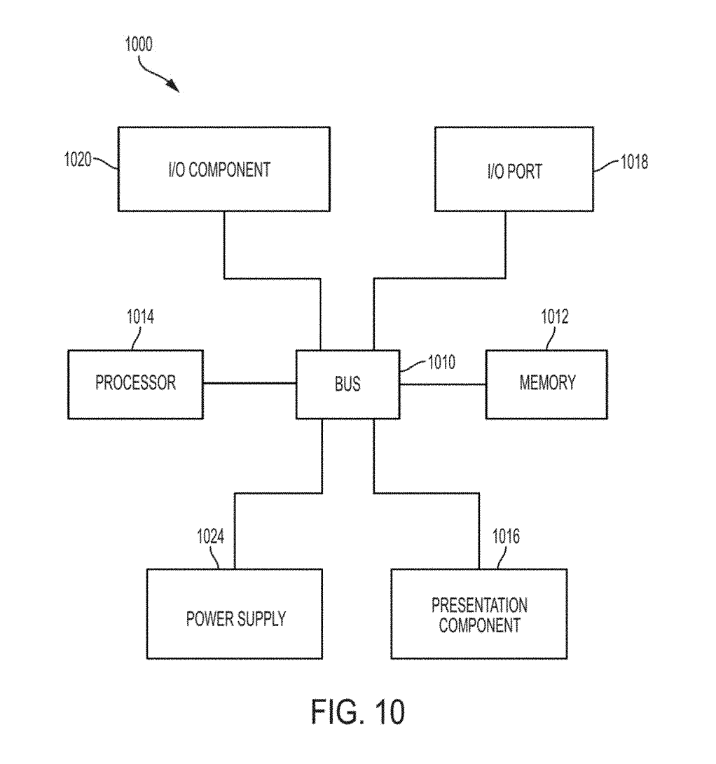

Any suitable computing device can be used to implement the computing devices 102, 104, 120 and methods/functionality described herein and be converted to a specific system for performing the operations and features described herein through modification of hardware, software, and firmware, in a manner significantly more than mere execution of software on a generic computing device, as would be appreciated by those of skill in the art. One illustrative example of such a computing device 1000 is depicted in FIG. 10. The computing device 1000 is merely an illustrative example of a suitable computing environment and in no way limits the scope of the present invention. A “computing device,” as represented by FIG. 10, can include a “workstation,” a “server,” a “laptop,” a “desktop,” a “hand-held device,” a “mobile device,” a “tablet computer,” or other computing devices, as would be understood by those of skill in the art. Given that the computing device 1000 is depicted for illustrative purposes, embodiments of the present invention may utilize any number of computing devices 1000 in any number of different ways to implement a single embodiment of the present invention. Accordingly, embodiments of the present invention are not limited to a single computing device 1000, as would be appreciated by one with skill in the art, nor are they limited to a single type of implementation or configuration of the example computing device 1000.

The computing device 1000 can include a bus 1010 that can be coupled to one or more of the following illustrative components, directly or indirectly: a memory 1012, one or more processors 1014, one or more presentation components 1016, input/output ports 1018, input/output components 1020, and a power supply 1024. One of skill in the art will appreciate that the bus 1010 can include one or more busses, such as an address bus, a data bus, or any combination thereof. One of skill in the art additionally will appreciate that, depending on the intended applications and uses of a particular embodiment, multiple of these components can be implemented by a single device. Similarly, in some instances, a single component can be implemented by multiple devices. As such, FIG. 10 is merely illustrative of an exemplary computing device that can be used to implement one or more embodiments of the present invention, and in no way limits the invention.

The computing device 1000 can include or interact with a variety of computer-readable media. For example, computer-readable media can include Random Access Memory (RAM); Read Only Memory (ROM); Electronically Erasable Programmable Read Only Memory (EEPROM); flash memory or other memory technologies; CDROM, digital versatile disks (DVD) or other optical or holographic media; magnetic cassettes, magnetic tape, magnetic disk storage or other magnetic storage devices that can be used to encode information and can be accessed by the computing device 1000.

The memory 1012 can include computer-storage media in the form of volatile and/or nonvolatile memory. The memory 1012 may be removable, non-removable, or any combination thereof. Exemplary hardware devices are devices such as hard drives, solid-state memory, optical-disc drives, and the like. The computing device 1000 can include one or more processors that read data from components such as the memory 1012, the various I/O components 1016, etc. Presentation component(s) 1016 present data indications to a user or other device. Exemplary presentation components include a display device, speaker, printing component, vibrating component, etc.

The I/O ports 1018 can enable the computing device 1000 to be logically coupled to other devices, such as I/O components 1020. Some of the I/O components 1020 can be built into the computing device 1000. Examples of such I/O components 1020 include a microphone, joystick, recording device, game pad, satellite dish, scanner, printer, wireless device, networking device, and the like.

As utilized herein, the terms “comprises” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about”, “generally”, and “approximately” are intended to cover variations that may existing in the upper and lower limits of the ranges of subjective or objective values, such as variations in properties, parameters, sizes, and dimensions. In one non-limiting example, the terms “about”, “generally”, and “approximately” mean at, or plus 10 percent or less, or minus 10 percent or less. In one non-limiting example, the terms “about”, “generally”, and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the present invention, and exclusive use of all modifications that come within the scope of the appended claims is reserved. Within this specification embodiments have been described in a way which enables a clear and concise specification to be written, but it is intended and will be appreciated that embodiments may be variously combined or separated without parting from the invention. It is intended that the present invention be limited only to the extent required by the appended claims and the applicable rules of law.

It is also to be understood that the following claims are to cover all generic and specific features of the invention described herein, and all statements of the scope of the invention which, as a matter of language, might be said to fall therebetween.

What is claimed is:

1. A method for verifying an intended or planned surgical procedure, the method comprising:
reviewing one or more obtained medical scans of a patient to select at least one apposite image;
receiving patient information associated with the selected apposite image, the patient information including an indication of sidedness of a procedure to be performed;
transforming the patient information into a scannable code to be placed on a limb of the patient corresponding to the sidedness of the procedure to be performed;
performing a validation based on the scannable code placed on a limb of the patient corresponding to the sidedness of the procedure to be performed and the selected apposite image to confirm procedure to be performed; and
determining whether to authorize the procedure based on the performed validation and indicating such authorization determination.

2. The method of claim 1, further comprising:
creating a wearable device having the scannable code.

3. The method of claim 2, wherein the wearable device comprises a bracelet.

4. The method of claim 2, further comprising:
placing the wearable device on a limb of the patient corresponding to the sidedness of the procedure to be performed.

5. The method of claim 1, wherein the patient information further comprises at least one of:
the procedure to be performed, a procedure code, a photograph of the patient, a name of the patient, and a medical record number of the patient.

6. The method of claim 1, wherein the scannable code is a Quick Response Code.

7. The method of claim 1, wherein the validation comprises:
performing a visual confirmation of patient;
scanning the scannable code to verify patient information; and
receiving, from a user, confirmation of sidedness of procedure to be performed.

8. The method of claim 7, wherein performing visual confirmation comprises:
taking a photograph of the patient holding up the limb with the scannable code indicating sidedness of the procedure to be performed; and
checking that the sidedness of the limb with the scannable code being held up in photo corresponds the sidedness of the procedure to be performed.

9. The method of claim 7, wherein performing visual confirmation of patient is performed using facial recognition software.

10. The method of claim 7, wherein the scanning of the scannable code to verify patient information comprises identifying sidedness of procedure to be performed.

11. The method of claim 7, wherein receiving, from a user, confirmation of sidedness for procedure to be performed comprises:
displaying the selected apposite image;
receiving a selection of a location of the displayed selected apposite image from the user; and
comparing the location of the selected apposite image selected by the user to sidedness of the procedure to be performed.

12. A surgical siting system for verifying an intended or planed surgical procedure, the system comprising:
a memory for storing data and instructions for execution; and
a processor configured to:
review one or more obtained medical scans of a patient to select at least one apposite image;
receive patient information associated with the selected apposite image, the patient information including an indication of sidedness of a procedure to be performed;
transform the patient information into a scannable code to be placed on a limb of the patient corresponding to the sidedness of the procedure to be performed;
perform a validation based on the scannable code placed on a limb of the patient corresponding to the sidedness of the procedure to be performed and the selected apposite image to confirm procedure to be performed; and
authorize the procedure based on the performed validation.

13. The system of claim 12, wherein the system is further configured to:
create a wearable device having the scannable code.

14. The system of claim 12, wherein the patient information further comprises at least one of: the procedure to be performed, a procedure code, a photograph of the patient, a name of the patient, and a medical record number of the patient.

15. The system of claim 12, wherein the scannable code is a Quick Response Code.

16. The system of claim 12, wherein the validation comprises:
performing visual confirmation of patient;
scanning the scannable code to verify patient information; and
receiving, from a user, confirmation of sidedness of procedure to be performed.

17. The system of claim 16, wherein configuring the processor to perform visual confirmation comprises configuring the processor to:
take a photograph of the patient holding up the limb with the scannable code indicating sidedness of the procedure to be performed; and
check that the sidedness of the limb with the scannable code being held up in photo corresponds the sidedness of the procedure to be performed.

18. The system of claim 16, wherein visual confirmation of patient is performed using facial recognition software.

19. The system of claim 16, wherein configuring the processor to scan the scannable code to verify patient information comprises configuring the processor to identify sidedness of procedure to be performed.

20. The system of claim 16, wherein configuring the processor to receive, from a user, confirmation of sidedness for procedure to be performed comprises configuring the processor to:
display the selected apposite image;
receive a selection of a location of the displayed selected apposite image from the user; and
compare the location of the selected apposite image selected by the user to sidedness of the procedure to be performed.

21. A method for verifying a surgical procedure, the method comprising:
providing a surgical siting system comprising a processor and memory;
providing a client device comprising a processor and memory in communication with the surgical siting system;
reviewing, on the user device, one or more obtained medical scans of a patient to select at least one apposite image;
storing a selected apposite image on the surgical siting system;
receiving, on the user device, patient information associated with the selected apposite image, the patient information including an indication of sidedness of a surgical procedure to be performed;
storing the patient information on the surgical siting system;
transforming, on the surgical siting system, the patient information into a scannable code to be placed on a limb of the patient corresponding to the sidedness of the procedure to be performed;
generating, using the client device, a wearable device having the scannable code provided by the surgical siting system;
placing the wearable device on a limb of the patient corresponding to the sidedness of the procedure to be performed;
performing, on the client device, a validation based on the scannable code placed on a limb of the patient corresponding to the sidedness of the procedure to be performed and the selected apposite image provided by the surgical siting system to confirm procedure to be performed; and
authorizing, on the client device, the procedure based on the performed validation.

22. The method of claim 21, wherein the patient information further comprises at least one of: the procedure to be performed, a procedure code, a photograph of the patient, a name of the patient, and a medical record number of the patient.

23. The method of claim 21, wherein the scannable code is a Quick Response Code.

24. The method of claim 21, wherein the validation comprises:
performing, on the client device, visual confirmation of patient;
scanning, on the client device, the scannable code to verify patient information stored on surgical siting system; and
receiving on the client device, from a user, confirmation of sidedness of procedure to be performed.

25. The method of claim 24, wherein performing visual confirmation comprises:
taking a photograph of the patient holding up the limb with the scannable code indicating sidedness of the procedure to be performed; and
confirming that the sidedness of the limb with the scannable code being held up in photo corresponds the sidedness of the procedure to be performed.

26. The method of claim 24, wherein performing visual confirmation of patient is performed using facial recognition software.

27. The method of claim 24, wherein the scanning of the scannable code to verify patient information comprises identifying sidedness of procedure to be performed stored on the surgical siting system.

28. The method of claim 24, wherein receiving, from a user, confirmation of sidedness for procedure to be performed comprises:
displaying, on the client device, the selected apposite image provided by the surgical siting system;
receiving, on the client device, a selection of a location of the displayed selected apposite image from the user; and
comparing the location of the selected apposite image selected by the user to sidedness of the procedure to be performed stored on the surgical siting system.
